SEQUENCE LISTING (8 sequences)

SEQ ID NO: 1 - DNA, 29 bp, Artificial Sequence
  Description of Artificial Sequence Synthetic primer
cgcgaagaaccttacctggttttgacatg

SEQ ID NO: 2 - DNA, 26 bp, Artificial Sequence
  Description of Artificial Sequence Synthetic primer
ggcaactaatgacaagggttgcgctc

SEQ ID NO: 3 - DNA, 25 bp, Artificial Sequence
  Description of Artificial Sequence Synthetic probe
aatgtcgttttccgcaaggacatat

SEQ ID NO: 4 - DNA, 25 bp, Artificial Sequence
  Description of Artificial Sequence Synthetic probe
cggaggagtgccttcgggagccgta

SEQ ID NO: 5 - DNA, 1544 bp, Neisseria gonorrhoeae
tgaacataagagtttgatcctggctcagattgaacgctggcggcatgctttacacatgcaagtcggacggcagcacagggaagcttgcttctcgggtggcgagtggcgaacgggtgagtaacatatcggaacgtaccgggtagcgggggataactgatcgaaagatcagctaataccgcatacgtcttgagagggaaagcaggggaccttcgggccttgcgctatccgagcggccgatatctgattagctggttggcggggtaaaggcccaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgtcagggaagaaaaggctgttgccaatatcggcggccgatgacggtacctgaagaataagcaccggctaactacgtgccagcagccgcggtaatacgtagggtgcgagcgttaatcggaattactgggcgtaaagcgggcgcagacggttacttaagcaggatgtgaaatccccgggctcaacccgggaactgcgttctgaactgggtgactcgagtgtgtcagagggaggtggaattccacgtgtagcagtgaaatgcgtagagatgtggaggaataccgatggcgaaggcagcctcctgggataacactgacgttcatgtccgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggttttgacatgtgcggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcggttgggcactctaatgagactgccggtgacaagccggaggaaggtggggatgacgtcaagtcctcatggcccttatgaccagggcttcacacgtcatacaatggtcggtacagagggtagccaagccgcgaggcggagccaatctcacaaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgcaaggagtccgcttaccacggtatgcttcatgactggggtgaagtcgtaacaaggtagccgtaggggaacctgcggctggatcacctcctttct

SEQ ID NO: 6 - DNA, 1548 bp, Chlamydia trachomatis
ctgagaatttgatcttggttcagattgaacgctggcggcgtggatgaggcatgcaagtcgaacggagcaattgtttcggcaattgtttagtggcggaagggttagtaatgcatagataatttgtccttaacttgggaataacggttggaaacggccgctaataccgaatgtggcgatatttgggcatccgagtaacgttaaagaaggggatcttaggacctttcggttaagggagagtctatgtgatatcagctagttggtggggtaaaggcctaccaaggctatgacgtctaggcggattgagagattggccgccaacactgggactgagacactgcccagactcctacgggaggctgcagtcgagaatctttcgcaatggacggaagtctgacgaagcgacgccgcgtgtgtgatgaaggctctagggttgtaaagcactttcgcttgggaataagagaagacggttaatacccgctggatttgagcgtaccaggtaaagaagcaccggctaactccgtgccagcagctgcggtaatacggagggtgctagcgttaatcggatttattgggcgtaaagggcgtgtaggcggaaaggtaagttagttgtcaaagatcggggctcaaccccgagtcggcatctaatactatttttctagagggtagatggagaaaagggaatttcacgtgtagcggtgaaatgcgtagatatgtggaagaacaccagtggcgaaggcgcttttctaatttatacctgacgctaaggcgcgaaagcaaggggagcaaacaggattagataccctggtagtccttgccgtaaacgatgcatacttgatgtggatggtctcaaccccatccgtgtcggagctaacgcgttaagtatgccgcctgaggagtacactcgcaagggtgaaactcaaaagaattgacgggggcccgcacaagcagtggagcatgtggtttaattcgatgcaacgcgaaggaccttacctgggtttgacatgtatatgaccgcggcagaaatgtcgttttccgcaaggacatatacacaggtgctgcatggctgtcgtcagctcgtgccgtgaggtgttgggttaagtcccgcaacgagcgcaacccttatcgttagttgccagcacttagggtgggaactctaacgagactgcctgggttaaccaggaggaaggcgaggatgacgtcaagtcagcatggcccttatgcccagggcgacacacgtgctacaatggccagtacagaaggtagcaagatcgtgagatggagcaaatcctcaaagctggccccagttcggattgtagtctgcaactcgactacatgaagtcggaattgctagtaatggcgtgtcagccataacgccgtgaatacgttcccgggccttgtacacaccgcccgtcacatcatgggagttggttttaccttaagtcgttgactcaacccgcaaggagagaggcgcccaaggtgaggctgatgactaggatgaagtcgtaacaaggtagccctaccggaaggtggggctggatcacctccttt

SEQ ID NO: 7 - DNA, 539 bp, Chlamydia trachomatis
agatatgtggaagaacaccagtggcgaaggcgcttttctaatttatacctgacgctaaggcgcgaaagcaaggggagcaaacaggattagataccctggtagtccttgccgtaaacgatgcatacttgatgtggatggtctcaaccccatccgtgtcggagctaacgcgttaagtatgccgcctgaggagtacactcgcaagggtgaaactcaaaagaattgacgggggcccgcacaagcagtggagcatgtggtttaattcgatgcaacgcgaaggaccttacctgggtttgacatgtatatgaccgcggcagaaatgtcgttttccgcaaggacatatacacaggtgctgcatggctgtcgtcagctcgtgccgtgaggtgttgggttaagtcccgcaacgagcgcaacccttatcgttagttgccagcacttagggtgggaactctaacgagactgcctgggttaaccaggaggaaggcgaggatgacgtcaagtcagcatggcccttatgcccagggcgacacacgtgctacaat

SEQ ID NO: 8 - DNA, 538 bp, Neisseria gonorrhoeae
agagatgtggaggaataccgatggcgaaggcagcctcctgggataacactgacgttcatgtccgaaagcgtgggtagcaaacaggattagataccctggtagtccacgccctaaacgatgtcaattagctgttgggcaacttgattgcttggtagcgtagctaacgcgtgaaattgaccgcctggggagtacggtcgcaagattaaaactcaaaggaattgacggggacccgcacaagcggtggatgatgtggattaattcgatgcaacgcgaagaaccttacctggttttgacatgtgcggaatcctccggagacggaggagtgccttcgggagccgtaacacaggtgctgcatggctgtcgtcagctcgtgtcgtgagatgttgggttaagtcccgcaacgagcgcaacccttgtcattagttgccatcattcggttgggcactctaatgagactgccggtgacaagccggaggaaggtggggatgacgtcaagtcctcatggcccttatgaccagggcttcacacgtcatacaat

(19) United States
(12) Patent Application Publication
(10) Pub. No.: US 2006/0292575 A1
(43) Pub. Date: Dec. 28, 2006

(54) Novel method for diagnosing pathogens of sexually transmitted diseases

(75) Inventor: George Chin-Sheng Chou, Tainan Hsien (TW)

Correspondence Address: TROXELL LAW OFFICE PLLC;ONE SKYLINE PLACE, SUITE 1404, 5205 LEESBURG PIKE, FALLS CHURCH, VA, 22041, US

(73) Assignee: AsiaGEN Corporation

(21) Appl. No.: 11/165,335

(22) Filed: Jun. 24, 2005

(51) Int. Cl. C12Q 1/68; C12M 1/34

(52) U.S. Cl. 435/6; 435/287.2

(57) ABSTRACT

The present invention provides a novel method for diagnosing various pathogens of sexually transmitted diseases from one test sample. The method comprises amplifying the DNA fragments of the various pathogens synchronously by using one primer set and identifying the amplified DNA fragments with specific probes. This invention also provides a designation process of the primer set and the probes. This invention further provides a kit for diagnosing of Neisseria gonorrhoeae and Chlamydia trachomatis.

### FIELD OF THE INVENTION

This invention relates to a method used for diagnosing the pathogens of sexually transmitted diseases. This invention further relates to polynucleotides for amplifying and identifying the pathogens of sexually transmitted diseases.

### BACKGROUND OF THE INVENTION

Sexually transmitted diseases (STDs) are a major health problem in the world. STDs exhibit a higher incidence and prevalence, an alarming rate of antimicrobial resistance, a higher rate of serious complications and interaction with human immunodeficiency virus (HIV) infection in developing countries. Failure to diagnose and treat traditional infections, such as gonorrhea, chlamydial infections and syphilis which can have deleterious effects during pregnancy and on the newborn, is also common in these countries.

Other complications especially in women, such as pelvic inflammatory disease, ectopic pregnancy, infertility and cervical cancer, are potent health and social problems. In most developing countries, the incidence and prevalence of STDs may be 20 times higher than those in developed countries. Point-prevalence studies are employed most widely in the developing world. Such information is useful but limited since it is not totally representative of the whole population as it is obtained mostly from high risk groups of individuals and/or patients. The developing world is a heterogeneous community, but it has at least one common feature, that the STDs in this community are expected to occur among those between 20 and 40 years of age, in contrast to the population of developed countries. The consequence of this is not only a higher absolute incidence of STDs in the developing countries but also a potentially worsening situation in the future.

STDs may be subdivided into curable and noncurable STDs. Curable STDs are Neisseria gonorrhoeae, Chlamydia trachomatis, Treponema pallidum, and Trichomonas vaginalis infections while non-curable STDs are of viral (HIV, herpes simplex virus (HSV), human papilloma virus (HPV) and hepatitis B virus origin.

The World Health Organization (WHO) has been responsible for surveying problems represented by STDs and HIV infection. It estimates an annual total of 333 million of new STDs infections in adults. The number of new cases is 12 million for syphilis, 62 million for gonorrhea, 89 million for chlamydial infections and 170 million for trichomoniasis, excluding genital papilloma virus infection which WHO itself had previously estimated at 30 million new cases per year and herpetic infection at 20 million. Genital ulcers show a relatively higher frequency among STDs, and chancroid followed by syphilis is a major cause of genital ulcers in the developing countries. STDs were concentrated in South East Asia with approximately 150 million new cases in 1995 and in sub-Saharan Africa with 64 million.

Chlamydia trachomatis (C. trachomatis or CT) and Neisseria gonorrhoeae (N. gonorrhoeae or NG) are two of the most common sexually transmitted infective agents. According to the data from WHO, the cases of infection of CT increase 4-10 millions per year, and the cases of infection of NG increase 3-4 millions per year, in global.

Chlamydia trachomatis (CT) is an obligate intracellular parasitic gram negative bacterium. There are 15 serotypes of CT, and different serotype induces different disease of human. The serotypes D ˜K are responsible for the venereal disease of male and female. In clinical, CT causes venereal lymphogranuloma, various inflammatory pathologies of the male and female urogenital systems, and trachoma, a chronic disease that affects 500 million people and can lead to blindness. When not precociously diagnosed and treated by adequate therapy, CT-induced urethritis and cervicitis may led to a variety of chronic inflammations, such as, e.g., vaginitis, salpingitis and pelvic inflammation which may result in sterility andextrauterine pregnancy. Furthermore, the new born from infected mothers may contract pulmonary and/or ocular infections during delivery.

There are many diagnostic methods for CT used in clinical research, such as cell culture, directly immunofluorescent antibody assay, and enzymatic immunoassay. However, following the development of technology, present diagnosis model are usage of DNA probe directly or combination of nucleic acid amplification and DNA probe confirmation. Although cell culture has been the standard method of diagnosis of CT, the scientific results proof that the sensitivity and specificity of using nucleic acid amplification and DNA probe are better than the traditional cell culture method.

N. gonorrhoeae (NG), the pathogen of gonorrhoea, a gram negative diplococcus, manifests itself as a purulent inflammation and swelling of the urethra in males. These symptoms occur in 80% of cases of infection, and occur in 2-7 days after infection. If left untreated, the infection can ascend and after several weeks produce symptoms of prostatitis.

In women, no or only slight symptoms occur in 80% of cases of infection. The infection primarily affects the cervix, but also the urethra. In 20% of cases of infection, the NG spreads to the fallopian tubes and can also lead to sterility. Since the course of the infections is often asymptomatic, many carriers contribute unknowingly to the spread of the disease.

Diagnosis of NG is using selective medium to isolate pathogen first, or using Gram's stain to observe the diplococci form. Though the clinical using of culture method presents good sensitivity, it is only under an appropriate situation. Improper storage and transportation of samples end up with pathogen loss and false positive result. Thus, the false positive result is caused by the factors including roughly sample technology, unsuitable sample material, and the pathogen-inhibitory components from human body. At present, the diagnostic model for N. gonorrhoeae used in common is the platform including non-cultured nucleic acid amplification and DNA probe confirmation.

### BRIEF DESCRIPTION OF THE DRAWINGS

FIG. 1 shows alignment result of 16S rRNA sequences from C. trachomatis and N. gonorrhoeae. NG: N. gonorrhoeae, and CT: C. trachomatis.

FIG. 2 shows the enlarged figure of the conserved region of the alignment result. NG: N. gonorrhoeae, and CT: C. trachomatis. Underline 1: the sequence for designing the forward primer; underline 2: the sequence for designing the reverse primer; and undulant underline: the sequence for designing the specific probe.

FIG. 3 shows the result of PCR of N. gonorrhoeae. The number labeled between two lanes (45, 50, 55, 60, 65, 70) means anneling temperature used in PCR program. NC: negative control, using the 0.22 μm filter filtered MQ water as PCR template; and Test: using the N. gonorrhoeae DNA as PCR template.

FIG. 4 shows part (A) PCR result by using 60° C. as annealing temperature. NC: negative control, using the 0.22 μm filter filtered MQ water as PCR template; NG and CT mean using the N. gonorrhoeae and C. trachomatis DNA as PCR template, respectively. Part (B) shows hybridization data of the PCR products with specific probe. The bar filled with diagonal is using NG-specific probe, and the checkered bar is using CT-specific probe. In the PCR condition, NC: negative control; PC: positive control, using NG and CT DNA as template, respectively; R-b: reverse primer labeled with biotin; and F-b: forward primer labeled with biotin.

FIG. 5 shows the specificity of hybridization. The bar filled with diagonal is using NG-specific probe, and the checkered bar is using CT-specific probe. In hybridization template, NC: negative control; NG: PCR product of N. gonorrhoeae; and CT: PCR product of C. trachomatis. Fig. A: the response time is 10 minutes and Fig. B: the response time is 20 minutes.

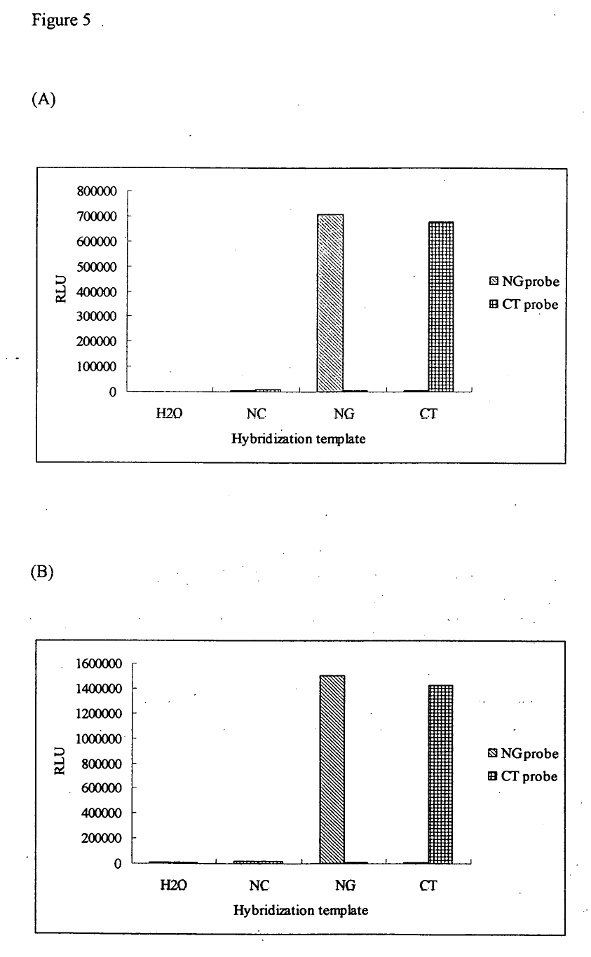

FIG. 6 shows part (A) the PCR result and part (B) shows hybridization data of the PCR products with specific probe. NC: negative control, using the 0.22 μm filter filtered MQ water as PCR template, TB: Mycobacterium tuberculosis, SA: Staphylococcus aureus, HI Haemophilus influenzae, PS: Streptococcus pneumonia, GP: Klebsiella pneumonia, KF2 Mycobacterium kansasii, RGM: Rapid grower Mycobacterium, MAC: Mycobacterium avium, NG: N. gonorrhoeae, and CT: C. trachomatis.

### BRIEF DESCRIPTION OF THE INVENTION

This invention provides a method for identifying various pathogens of sexually transmitted diseases from one test sample comprises: (a) amplifying DNA fragments of the various pathogens synchronously by one primer set; (b) hybridizing the amplified DNA fragments with a specific probe; and (c) isolating and confirming the hybrid products. This invention also provides a process for designing a primer set for amplifying DNA fragments of various pathogens of sexually transmitted diseases synchronously comprises: (a) selecting a conserved region of genome between the microorganisms; (b) aligning the sequences of the conserved region of the microorganisms; (c) selecting a target fragment from the conserved region having the sequence in two ends are identical between the microorganisms, and having different sequences for identifying the microorganisms; (d) choosing the sequence in two ends of the target fragment to be primer set; (e) defining forward and reverse primers, wherein the sequence of the forward primer from one microorganism is the same with the forward primer from another microorganism, and the sequence of the reverse primer from one microorganism is the same with the reverse primer from another microorganism; and (f) choosing the different part of the target fragment to be a probe. This invention also provides a primer set for amplifying DNA fragments of Neisseria gonorrhoeae and Chlamydia trachomatis consisting of 5′-CGCGMG MCCTTACCTGGTTTTGACATG-3′ or 5′-GGCMCTMTGACAAGGGTTGCGCTC-3′. This invention also provides a probe for identifying an amplified DNA fragment from Neisseria gonorrhoeae and/or Chlamydia trachomatis consisting of: (a) 5′-MTGTCGTTTTCCGCMGGACATAT-3′ for Chlamydia trachomatis; and (b) 5′-CGGAGGAGTGCCTTCGGGAGCCGTA-3′ for Neisseria gonorrhoeae. This invention further provides a kit for detecting Neisseria gonorrhoeae and Chlamydia trachomatis comprising: a primer set used to amplify DNA of a sample; and probes used to identify the amplified DNA fragment.

### DETAILED DESCRIPTION OF THE INVENTION

Definitions

The following terms, as used in this disclosure and claims, are defined as:

Nucleotide:

A subunit of a nucleic acid consisting of a phosphate group, a 5′ carbon sugar and a nitrogen containing base. In RNA the 5′ carbon sugar is ribose. In DNA, it is a 2-deoxyribose. The term also includes analogs of such subunits.

Nucleic Acid Probe:

A single stranded nucleic acid sequence that will combine with a complementary single stranded target nucleic acid sequence to form a double-stranded molecule (hybrid). A nucleic acid probe may be an oligonucleotide or a nucleotide polymer.

Hybridization:

The process by which two complementary strands of nucleic acids combine to form double stranded molecules (hybrids).

Probe Specificity:

Characteristic of a probe which describes its ability to distinguish between target and non-target sequences. According to sequence and assay conditions, probe specificity may be absolute (i.e., probe able to distinguish between target organisms and any non-target organisms), or it may be functional (i.e., probe able to distinguish between the target organism and any other organism normally present in a particular sample). Many probe sequences can be used for either broad or narrow specificity depending on the conditions of use.

Variable region: nucleotide polymer which differs by at least one base between the target organism and non-target organisms contained in a sample.

Conserved region: a region which is not variable.

This invention relates to a method for identifying various pathogens of sexually transmitted diseases (STD) comprises: (a) amplifying DNA fragments of the various pathogens synchronously by one primer set, (b) hybridizing the amplified DNA fragments with different specific probes; and (c) confirming the hybrid products. The pathogens of sexually transmitted diseases are selected from the group consisting of: bacteria, virus, fungi, rickettsial and Chlamydia. Using this method, the pathogen can be detected from one test sample.

The method comprises only once DNA amplification process and specific hybridization process. The DNA amplification process comprises a novel primer set, which is designed from a conserved region between the genomes of the various pathogens and used for amplifying DNA fragments all pathogens of STDs synchronously. The conserved region is selected from the group consisting of 5S rRNA genes, 16S rRNA genes, 23S rRNA genes, 5.0S rRNA genes, 5.8S rRNA genes, 18S rRNA genes, 28S rRNA genes, 16S like rRNA genes, and 23S like rRNA genes. The specific hybridization process comprises several specific probes, which are designed from the variable region of the conserved region and used for identifying the amplified DNA products.

The designation of the primer and the probe comprises several steps described as follows:

The process for designing a primer set for amplifying DNA fragments of various pathogens of sexually transmitted diseases comprises: (a) selecting a conserved region of genomes between the various pathogens, (b) aligning the sequences of the conserved region of the various pathogens, (c) selecting a target fragment from the conserved region having the sequence in two ends are identical between the various pathogens, and having different sequences for identifying the various pathogens, (d) choosing the sequence in two ends of the target fragment to be primers, (e) defining forward and reverse primers, wherein the sequence of the forward primer from one pathogen is the same with the forward primer from another pathogens, and the sequence of the reverse primer from one pathogen is the same with the reverse primer from another pathogens; and (f) choosing the different part of the target fragment to be a probe specific to the pathogen.

First, selecting several pathogens which we want to detect from the pathogen of STDs, for example: Chlamydia trachomatis/Neisseria gonorrhoeae, Chlamydia trachomatis/Treponema pallidum, or Chlamydia trachomatis/Neisseria gonorrhoeae/Treponema pallidum. The pathogens of sexually transmitted diseases are selected from the group consisting of: bacteria, virus, fungi, rickettsial and Chlamydia.

Aligning the nucleotide sequences of the selected pathogens is by use of bio-information software such as DNAsis. For saving aligning time, we can select some genes or some DNA regions which are well-known existing highly conserved sequences, such as rRNA genes. With the exception of viruses, all prokaryotic organisms contain rRNA molecules including 5S rRNA, 16S rRNA, and a larger rRNA molecule known as 23S rRNA. Eukaryotes are known to have 5.0S, 5.8S, 18S and 28S rRNA molecules or analogous structures. (The term “16S like” sometimes is used to refer to the rRNA found in the small ribosomal subunit, including 18S and 17S rRNA. Likewise the term “23S like” rRNA sometimes is used to refer to the rRNA found in the large ribosomal subunit. “5S like” rRNA sometimes is used to refer to rRNA found in the large ribosomal subunit. 5.8S rRNA is equivalent to the 5′ end of the 23S like rRNA.) These rRNA molecules contain nucleotide sequences which are highly conserved among all organisms thus far examined.

Notwithstanding the highly conserved nature of rRNA, it is discovered that a number of regions of the rRNA molecule which can vary in sequence, can vary even between closely related species and can, therefore, be utilized to distinguish between such organisms. Differences in the rRNA gene are not distributed randomly across the entire gene, but rather are clustered into specific regions. The degree of conservation also varies, creating a unique pattern of conservation across the ribosomal RNA subunits. The degree of variation and the distribution can be analyzed to locate target sites for diagnostic probes.

The selected DNA sequences of the pathogens are aligned to screen conserved regions present in all selected pathogens. From these screened conserved regions, we select one conserved region having the sequences in the two ends are identical between the selected pathogens, and having variable regions between the two ends. The sequences of the two ends of the conserved regions are unnecessary to be identical to each other, more precisely to say, the sequence of one end can be different with the other end. However, the sequences of one end of the conserved region have to be identical between the selected pathogens.

From the two end of the conserved region, we select suitable length of nucleotides to design a primer set, including forward primer and reverse primer. Defining forward and reverse primer, wherein the sequence of the forward primer from one pathogen is the same with the forward primer from another pathogen, and the sequence of the reverse primer from one pathogen is the same with the reverse primer from another pathogen. Base on the described steps, the designed primer set can be used to amplify the all selected pathogens.

From the variable regions between the two ends of the conserved region, we select a unique nucleotide fragment of the variable regions, which is specific to each selected pathogen. The suitable length of the unique nucleotide fragment is chosen to design the probes. Each designed probe is highly specific to each pathogen. Thus, after amplifying the DNA fragments of the selected pathogens from test sample, hybridizing the specific probe and the nucleotide sequence complementary to the amplified DNA products, we can diagnose whether the probe-specific pathogen exists in the test sample.

Amplification procedures are well-known in the art and include, but are not limited to, polymerase chain reaction (PCR), TMA, rolling circle amplification, nucleic acid sequence based amplification (NASBA), and strand displacement amplification (SDA). One skilled in the art will understand that for use in certain amplification techniques the primers may need to be modified, for example, for SDA the primer comprises additional nucleotides near its 5′ end that constitute a recognition site for a restriction endonuclease. Similarly, for NASBA the primer comprises additional nucleotides near the 5′ end that constitute an RNA polymerase promoter. Polynucleotides thus modified are considered to be within the scope of the present invention.

As is well known in the art, certain criteria need to be taken into consideration when selecting a primer for an amplification reaction. For example, when a primer pair is required for the amplification reaction, the primers should be selected such that the likelihood of forming 3′ duplexes is minimized, and such that the melting temperatures(TM) are sufficiently similar to optimize annealing to the target sequence and minimize the amount of non-specific annealing.

In this context, the polynucleotides according to the present invention are provided in combinations that can be used as primers in amplification reactions to specifically amplify target nucleic acid sequences.

The amplification method of the present invention generally comprises (a) forming a reaction mixture comprising nucleic acid amplification reagents, at least one primer/probe set of the present invention, and a test sample suspected of containing at least one target sequence and (b) subjecting the mixture to amplification conditions to generate at least one copy of a nucleic acid sequence complementary to the target sequence.

Step (b) of the above methods can be repeated any suitable number of times (prior to step (c) in the detection method), e.g., by thermal cycling the reaction mixture between 10 and 100 times, typically between about 20 and about 60 times, more typically between about 25 and about 45 times.

Nucleic acid amplification reagents include reagents which are well known and may include, but are not limited to, an enzyme having at least polymerase activity, enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

Amplification conditions are conditions that generally promote annealing and extension of one or more nucleic acid sequences. It is well known that such annealing is dependent in a rather predictable manner on several parameters, including temperature, ionic strength, sequence length, complementarity, and G: C content of the sequences. For example, lowering the temperature in the environment of complementary nucleic acid sequences promotes annealing. For any given set of sequences, melt temperature, or Tm, can be estimated by any of several known methods. Typically, diagnostic ap° C. plications utilize hybridization temperatures that are about 10° C. (e.g, 2° C. to 18° C.) below the melt temperature. Ionic strength or “salt” concentration also impacts the melt temperature, since small cations tend to stabilize the formation of duplexes by negating the negative charge on the phosphodiester backbone. Typical salt concentrations depend on the nature and valency of the cation but are readily understood by those skilled in the art. Similarly, high G:C content and increased sequence length are also known to stabilize duplex formation because G:C pairings involve 3 hydrogen bonds where A:T pairs have just two, and because longer sequences have more hydrogen bonds holding the sequences together. Thus, a high G:C content and longer sequence lengths impact the hybridization conditions by elevating the melt temperature.

Upon selecting sequences for a given diagnostic application, the G:C content and length will be known and can be accounted for in determining precisely what hybridization conditions will encompass. Since ionic strength is typically optimized for enzymatic activity, the only parameter left to vary is the temperature. Generally, the hybridization temperature is selected close to or at the Tm of the primers or probe. Thus, obtaining suitable hybridization conditions for a particular primer/probe set is well within ordinary skill of one practicing this art.

Specific amplicons produced by amplification of target nucleic acid sequences using the polynucleotides of the present invention, as described above, can be detected by a variety of methods known in the art. For example, one or more of the primers used in the amplification reactions may be labeled such that an amplicon can be directly detected by conventional techniques subsequent to the amplification reaction. Alternatively, a probe consisting of a labeled version of one of the primers used in the amplification reaction, or a third polynucleotide distincts from the primer sequences that has been labeled and is complementary to a region of the amplified sequence, can be added after the amplification reaction is complete. The mixture is then submitted to appropriate hybridization and wash conditions and the label is detected by conventional methods.

The amplification product produced as above can be detected during or subsequently to the amplification of the target sequence. Gel electrophoresis can be employed to detect the products of an amplification reaction after its completion. Alternatively, amplification products are hybridized to probes, then separated from other reaction components and detected using microparticles and labeled probes.

For convenient utilization, the primers and probes are labeled with detectable component or molecules. The probe is labeled with a magnetic particle, become a magnetic probe. The primer, including forward primer, reverse primer or both, is labeled by a bioactive component such as biotin. The amplified DNA product hybridized with magnetic probe to form a hybrid complex, and this hybrid complex has affinity with a magnetic material, so that the hybrid complex can be easily separated from unbound DNA products by using a magnetic rack. The hybrid complex can be further quantitative by using the primer label. For example, if the primer is labeled with biotin, adding avidin, the substrate of biotin, into the isolated hybrid complex. The hybrid complex can be further quantitated by reading luminescence by Luminometer.

In one embodiment of the present invention, a novel primer set used for amplifying the DNA fragments of Chlamydia trachomatis and Neisseria gonorrhoeae synchronously from one test sample. The primer set for amplifying DNA fragments of Neisseria gonorrhoeae and Chlamydia trachomatis consisting of: 5′-CGCGMGAACCTTACCTGGTTTTGACATG-3′ and 5′-GGCMCTAATGACAAGGGTTGCGCTC-3′. The primer set is designed from a conserved region of the genome of Neisseria gonorrhoeae and Chlamydia trachomatis, in which conserved region is from 16S rRNAgene. The primer is labeled with a bioactive component. In better embodiment, the component is biotin.

The primer set designed by the process describe as follows: selecting 16S rRNA gene, which is well-known the highly conserved region of genome; aligning the nucleotide sequences of the 16S rRNA gene to screen the conserved region; selecting a target fragment from the conserved region of 16S rRNA gene having the sequence in two ends are identical between C. trachomatis and N. gonorrhoeae, and having variable region which is different sequences for identifying C. trachomatis and N. gonorrhoeae; and selecting the suitable sequence length in two ends of the target fragment of the conserved region to design the primer set. The primer set including a forward primer and a reverse primer. Defining the forward and reverse primers, wherein the sequence of the forward primer from C. trachomatis is the same with the forward primer from N. gonorrhoeae, and the sequence of the reverse primer from C. trachomatis is the same with the reverse primer from N. gonorrhoeae.

In one embodiment of the present invention, a probe for identifying an amplified DNA fragment from Neisseria gonorrhoeae and/or Chlamydia trachomatis consisting of: (a) 5′-MTGTCGTTTTCCGCA AGGACATAT-3′ for Chlamydia trachomatis; and (b) 5′-CGGAGGAGTGCCTTCGGGAGCCGTA-3′ for Neisseria gonorrhoeae. The probe is labeled with a magnetic particle. The probe is designed from a variable region of the conserved region of the genome of Neisseria gonorrhoeae and Chlamydia trachomatis, wherein the region has different DNA sequence for identifying Neisseria gonorrhoeae and Chlamydia trachomatis. The conserved region is from 16S rRNA gene. The variable region of C. trachomatis is specific to C. trachomatis, and the variable region of N. gonorrhoeae is specific to N. gonorrhoeae. The selected variable region is located in the conserved region, which comprises the two ends for designing the primer set, so that the selected variable region is amplified by using the primer set. A suitable sequence length of the variable region is selected to design the specific probes.

In one embodiment of the present invention, a kit for detecting Neisseria gonorrhoeae and Chlamydia trachomatis comprising: (a) a primer set used to amplify DNA of a sample consisting of 5′-CGCGMG AACCTTACCTGGTTTTGACATG-3′ and 5′-GGCMCTMTGACMGGGTT GCGCTC-3′; and (b) probes used to identify the amplified DNA fragment comprising: 5′-CGGAGGAGTGCCTTCGGGAGCCGTA-3′ for Neisseria gonorrhoeae, and 5′-MTGTCGTTTTCCGCMGGACATAT-3′ for Chlamydia trachomatis. The primer set can be used to amplify the DNA fragments of C. trachomatis and Neisseria gonorrhoeae simultaneously. The one of probes is specific to C. trachomatis, and another is specific to N. gonorrhoeae. The primer is labeled with biotin, and the probe is labeled with a magnetic particle as a magnetic probe. The kit further comprises a magnetic rack for separating the hybrid complex. The kit further comprises a substrate (such as biotin) of enzyme for quantitating the hybrid complex.

The process of utilizing the kit for diagnosis of C. trachomatis and/or N. gonorrhoeae as described as follows; obtaining the proper sample from patient, in clinical, or any source containing the pathogens; amplifying the target DNA fragments from the treated sample by using the biotin-labeled primer set; adding the magnetic probe specific to C. trachomatis (or N. gonorrhoeae) into the amplified products to hybridize the specific target; using the magnetic rack to isolate the hybrid complex and separate unbound amplified products; confirming the hybrid complex by adding avidin-Horse radish peroxidase and its substrate, and reading the luminescence by Luminmeter.

This invention provides a convenient method to diagnosing the pathogens of STDs. The kit prepared based on the former method provides as easy and economic tool for diagnosis of the pathogens of STDs, especially to the most two common pathogens, C. trachomatis and N. gonorrhoeae. The kit is not only saving the time of detection, but also presenting the high specificity and sensitivity to the pathogens. Most interestingly, the kit is able to detect pathogens from both urine and ordinary clinical samples (swab).

### EXAMPLE

### Example 1

### Sequence Alignment and Primer Probe Search

The sequences of 16S rRNAgene of N. gonorrhoeae and C. trachomatis were analyzed by software DNAsis to screen conserved regions of them. The primer was designed based on the conserved region, and the probe was further designed based on the specific sequences among the conserved region.

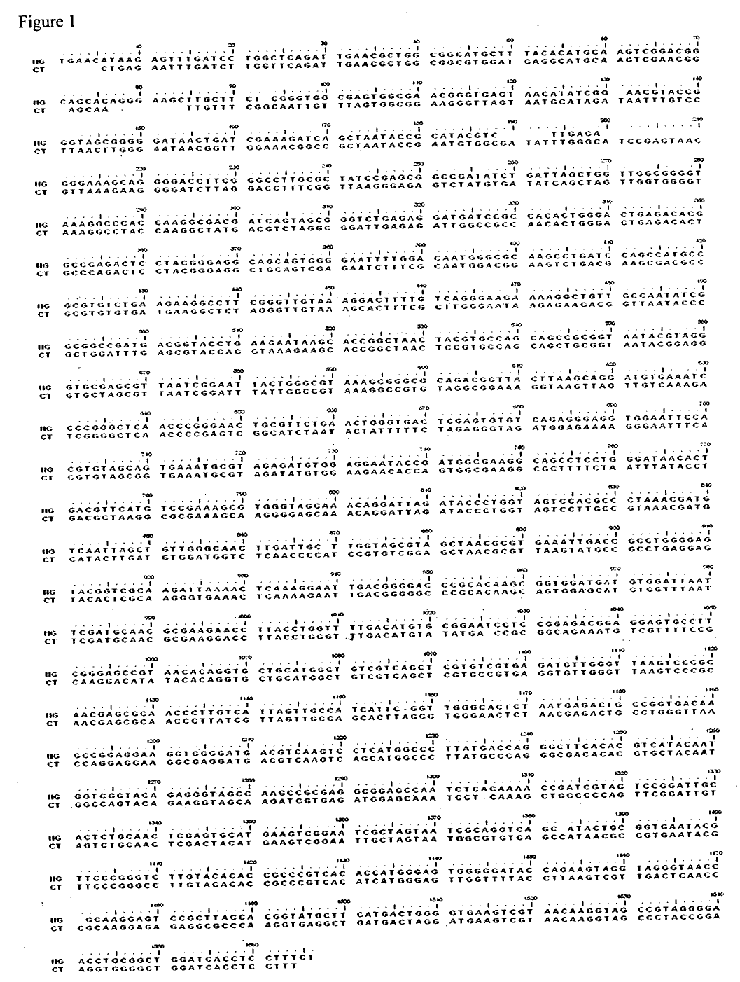

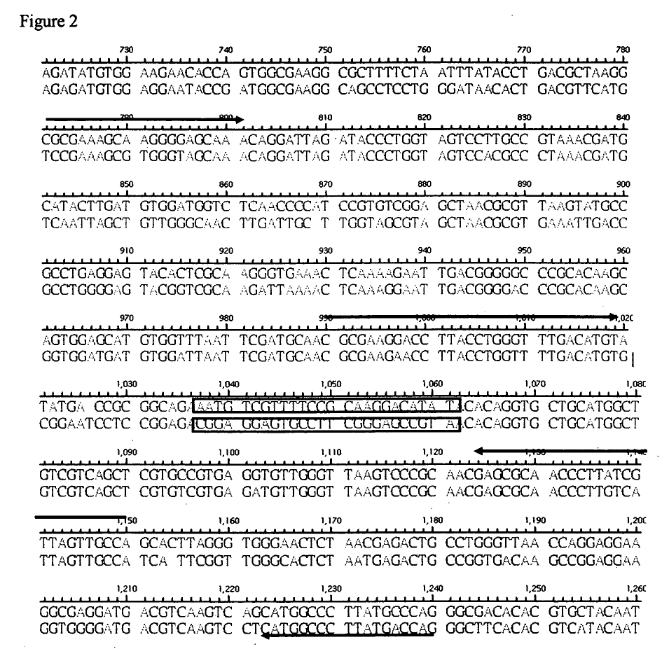

The DNA alignment result as shown in FIG. 1, there presented 72.4% homology between N. gonorrhoeae and C. trachomatis. A conserved region was screened from 957 bp to 1131 bp. The enlarged figure of this conserved region was showed in FIG. 2. The two fragments, underline 1 and underline 2, are identical sequences between N. gonorrhoeae and C. trachomatis. The sequences of underline 1 and underline 2 were designed as forward probe (F) and reverse probe (R), respectively. Further, the fragment with undulant underline was specific to each pathogen. The sequences of the fragment were designed as specific probe.

### Example 2

### Preparation of Clinical Sample

SWAB: The sampled swab was placed into 15 ml centrifugal tube containing 2 ml 1×PBS, then vortex for 1 min to separate the adherent components. The sampled swab was clipped by burnt tweezers, and all liquid was squeezed out from swab. The 15 ml tube was centrifuged in 3000 rpm for 5 min. The suspension was removed. 100 μl PCR buffer, Tween 20 (0.45%), and proteinase K were added, then mix completely. Transferred to 1.5 ml Eppendorf and sat at 55° C. for 1 hour, then held in 95° C. for 10 min to remove proteinase K activities. The final mixture could be proceed to PCR directly, or stored at −20° C. until use.

URINE: The former section of urine was collected about 20˜30 ml from patient. The urine was placed into 50 ml centrifugal tube. The tube was centrifuged in 12,000 rpm for 30 min and then the suspension was removed. 100 μl PCR buffer, Tween 20 (0.45%), and proteinase K were added and mixed completely. Transferred to 1.5 ml Eppendorf and sat at 55° C. for 1 hour, then held in 95° C. for 10 min to remove proteinase K activities. The final mixture could be proceed to PCR directly, or stored at −20° C. until use.

### Example 3

### Polymerase Chain Reaction

The sequences of the primer set were 5′-CGCGAAGAACCTTACCTGGTTTTG ACATG-3′ (F) and 5′-GGCAACTAATGACAAGGGTTGCGCTC-3′ (R). 50 μl PCR reactions contained 1×PCR buffer containing 1.5 mM MgCl2 (GeneTeks Bioscience Inc.), 2 μM each primer, 2 mM of each dNTP (ProTech), 2 U Taq polymerase and 2 μl template. The thermal cycling conditions were 94° C. for 5 min and 30 cycles of 94° C. for 45 s, 60° C. for 30 s, and 72° C. for 30 s with final extension at 72° C. for 10 min in a MyCycler Thermal Cycler (Bio-Rad). 160 bp PCR products were electrophoresed through a 2% agarose gel containing 1 μg/ml ethidium bromide.

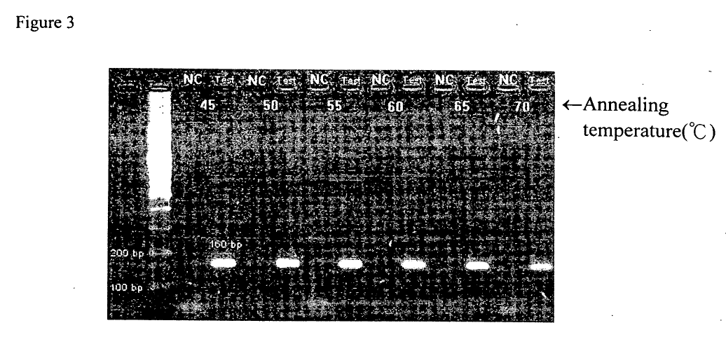

To screen the proper annealing temperature of the primers for better nucleic acid replication in PCR, the DNA of N. gonorrhoeae was used and six different annealing temperatures including 45° C., 50° C., 55° C., 60° C., 65° C., and 70° C. were selected to test. The result showed in FIG. 3, the suitable annealing temperature of the primer set was wide-range. Avoid contamination and non-specific replication occurred in PCR, 60° C. was selected to be the annealing temperature of PCR in later experiments.

### Example 4

### Hybridization and Signal Detection

The sequences of probe for C. trachomatis were 5′-AATGTCGTTTTC CGCAAGGACATAT-3′. The sequences of probe for N. gonorrhoeae were 5′-CGGAGGAGTGCCTTCGGGAGCCGT-3′. 275 μl Hybridization buffer, 15 μl 1 self coupled magnetic probe (Seradyn Inc.), and 10 μl PCR product were added into hybridization tube (Ht), then mix completely by vortex. The Ht was kept at 95° C. dry bath for 5 min. After the Ht was hold in 50° C. dry bath for 20 min, it was transferred to the magnetic well of dry bath and the hybridization buffer was removed by aspiration. Wash buffer (pre-heat at 50° C.) was added to each tube, vortex and put back to magnetic tube to hold for 3˜5 min, then the wash buffer was removed by aspiration. The former step was repeated once. The freshly prepared blocking buffer containing 1: 4,000 diluted SA-HRP (Pierce Biotechnology, Inc.) was added and sat at room temperature for 20 min and avoid light. The Ht was put into magnetic rack and the solution was removed by aspiration. 0.5% PBST was added, and the Ht was vortexed and put back to magnetic rank. The solution was removed by aspiration. The former step was repeated once. Proper volume of 1×PBS was used in each tube to re-suspend magnetic beads by vortex. The substrate (Pierce Biotechnology, Inc.) was added to each tube, and the luminescence was read by Luminometer.

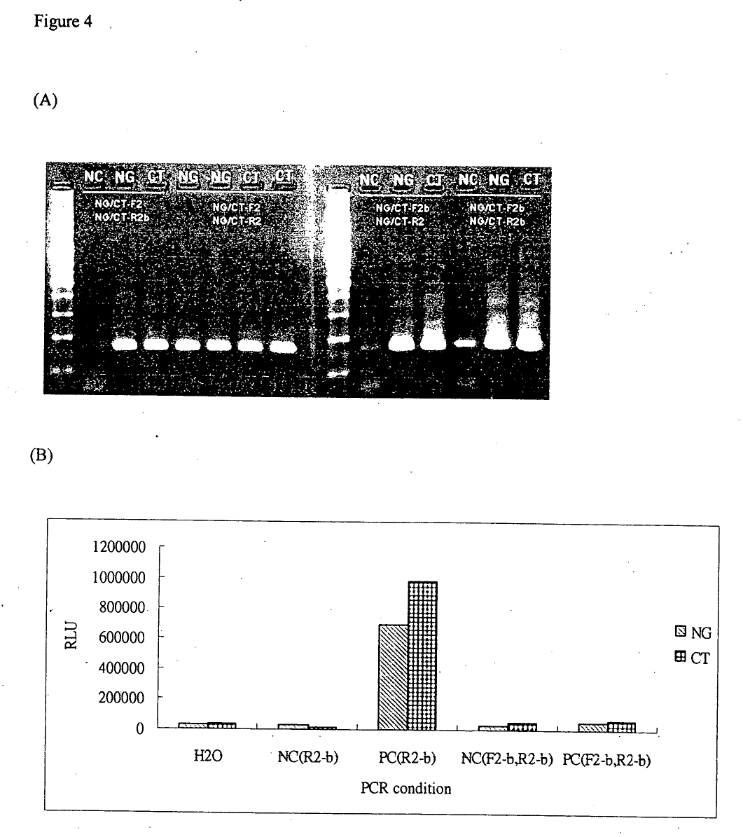

The result showed in FIG. 4A was the PCR products of N. gonorrhoeae and C. trachomatis. The amplified DNA products of the two pathogens were about 160 bp, and there is no other band on the electrophoresis gel. The result showed in FIG. 4B was the hybridization result. It presented the significant signal only in the sample containing the target amplified DNA products.

### Example 5

### Non-Cross Reactions Between in Two Specific Probes

As shown in FIG. 4, the NG-specific probe recognized the PCR product of N. gonorrhoeae, and didn't recognize the PCR product of C. trachomatis. The CT-specific probe only recognized the PCR product of C. trachomatis, didn't recognized the PCR product of N. gonorrhoeae. Whatever response time is 10 minutes or 20 minutes, the significant signal can be detected.

### Example 6

### Specificity of the Detection System

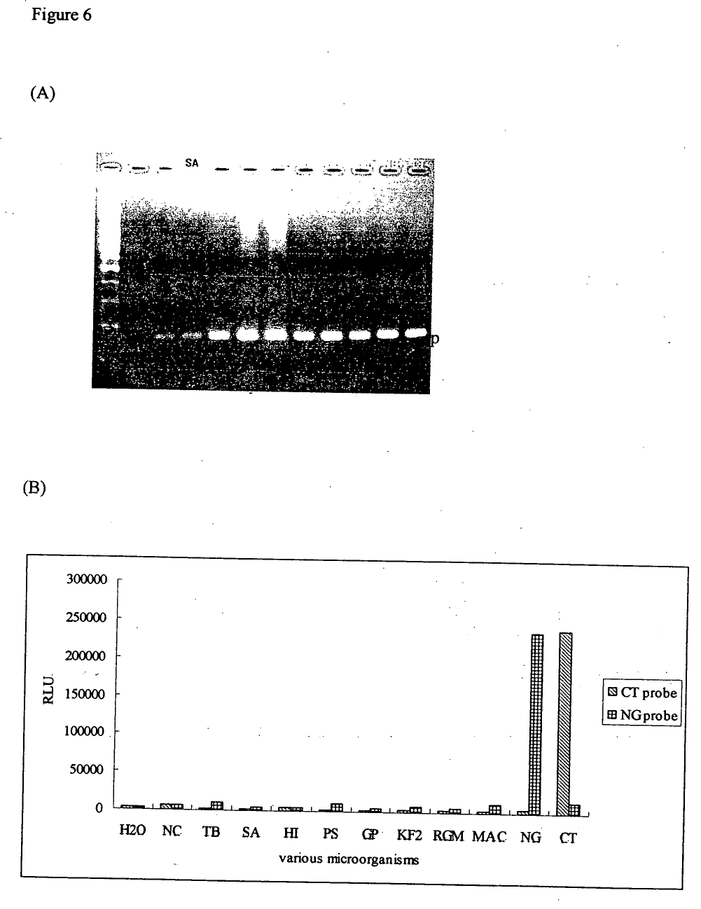

The primer set (primer F and R) and the two specific probes (CT probe and NG probe) were used to set up a detection system. To assay the specificity of the kit, eight bacterial strains were selected, including Mycobacterium tuberculosis, Staphylococcus aureus, Haemophilus influenzae, Streptococcus pneumoniae, Klebsiella pneumoniae, Mycobacterium kansasii, Rapid grower Mycobacterium, and Mycobacterium avium. DNA of these strains was used as PCR templates, and the products were hybridized with the two specific probes. FIG. 6 (A) showed the PCR products, the primer set of this system amplified specific DNA fragments in these strains. Further probe confirmation results were showed in FIG. 6 (B), the CT probe only responded with the C. trachomatis PCR product and the NG probe only responded with the N. gonorrhoeae PCR product. The eight bacterial strains presented unobvious results of hybridization. The hybridization results provided an evidence of specificity of this detection system.

What is claimed is:

1. A method for identifying various pathogens of sexually transmitted diseases comprises:
(a) amplifying DNA fragments of the various pathogens synchronously by one primer set;
(b) hybridizing the amplified DNA fragments with different specific probes; and
(c) confirming the hybrid products.

2. The method as claimed in claim 1, wherein the pathogens of sexually transmitted diseases are selected from the group consisting of: bacteria, virus, fungi, rickettsial and Chlamydia.

3. The method as claimed in claim 1, wherein the primer set is designed from a conserved region between the genomes of the various pathogens.

4. The method as claimed in claim 3, wherein the conserved region is selected from the group consisting of 5S rRNA genes, 16S rRNA genes, 23S rRNA genes, 5.0S rRNA genes, 5.8S rRNA genes, 18S rRNA genes, 28S rRNA genes, 16S like rRNA genes, and 23S like rRNA genes.

5. The method as claimed in claim 1, wherein the specific probe is designed from the variable region of the conserved region as in claim 3.

6. A process for designing a primer set for amplifying DNA fragments of various pathogens of sexually transmitted diseases comprises:
(a) selecting a conserved region of genomes between the various pathogens;
(b) aligning the sequences of the conserved region of the various pathogens;
(c) selecting a target fragment from the conserved region having the sequence in two ends are identical between the various pathogens, and having different sequences for identifying the various pathogens;
(d) choosing the sequence in two ends of the target fragment to be primers;
(e) defining forward and reverse primers, wherein the sequence of the forward primer from one pathogen is the same with the forward primer from another pathogens, and the sequence of the reverse primer from one pathogen is the same with the reverse primer from another pathogens; and
(f) choosing the different part of the target fragment to be a probe specific to the pathogen.

7. The process as claimed in claim 6, wherein alignment in the step (b) is by use of bio-information software DNAsis.

8. The process as claimed in claim 6, wherein the pathogens of sexually transmitted diseases are selected from the group consisting of: bacteria, virus, fungi, rickettsial and Chlamydia.

9. A primer set for amplifying DNA fragments of Neisseria gonorrhoeae and Chlamydia trachomatis consisting of:

| | |
|---|---|
| 5′-CGCGAAGAACCTTACCTGGTTTTGACATG-3′ | (SEQ ID NO: 1) |
| and | |
| 5′-GGCAACTAATGACAAGGGTTGCGCTC-3′. | (SEQ ID NO: 2) |

10. The primer set as claimed in claim 9, which is designed from a conserved region of the genome of Neisseria gonorrhoeae and Chlamydia trachomatis.

11. The primer set as claimed in claim 10, wherein the conserved region is from 16S rRNAgene.

12. The primer set as claimed in claim 9, which is labeled with a bioactive component.

13. The primer set as claimed in claim 12, wherein the component is biotin.

14. A probe for identifying an amplified DNA fragment from Neisseria gonorrhoeae and/or Chlamydia trachomatis consisting of: (a) 5′-AATGTCGTTTTCCGCAAGGACATAT-3′ (SEQ ID NO: 3) for Chlamydia trachomatis; and (b) 5′-CGGAGGAGTGCCTTCGGGAGCCGTA-3′ (SEQ ID NO: 4) for Neisseria gonorrhoeae.

15. The probe as claimed in claim 14, which is designed from a variable region of the conserved region of the genome of Neisseria gonorrhoeae and Chlamydia trachomatis, wherein the region has different DNA sequence for identifying Neisseria gonorrhoeae and Chlamydia trachomatis.

16. The primer as claimed in claim 15, wherein the conserved region is from 16S rRNAgene.

17. The probe as claimed in claim 14, which is labeled with a magnetic particle.

18. A kit for detecting Neisseria gonorrhoeae and Chlamydia trachomatis comprising
(a) a primer set used to amplify DNA of a sample consisting of 5′-CGCGMGAACCTTACCTGGTTTTGACATG-3′ (SEQ ID NO: 1) and 5′-GGCAACTAATGACAAGGGTTGCGCTC-3′ (SEQ ID NO: 2); and
(b) probes used to identify the amplified DNA fragment comprising 5′-CGGAGGAGTGCCTTCGGGAGCCGTA-3′ (SEQ ID NO: 4) for Neisseria gonorrhoeae, and 5′-AATGTCGTTTTCCGCMGGACATAT-3′ (SEQ ID NO: 3) for Chlamydia trachomatis.

19. The kit as claimed in claim 18, wherein the primer set is labeled with a bioactive component.

20. The kit as claimed in claim 19, wherein the component is biotin.

21. The kit as claimed in claim 18, wherein the probe is labeled with a magnetic particle.

22. The kit as claimed in claim 18, further comprising a magnetic rack.

23. The kit as claimed in claim 18, further comprising a substrate of enzyme for quantitating the hybrid complex.
